(19) United States
(12) Patent Application Publication
(10) Pub. No.: US 2026/0315048 A1
(43) Pub. Date: Oct. 15, 2026

(54) MEDICAL SAMPLING DEVICE AND SYSTEM COMPRISING THE MEDICAL SAMPLING DEVICE

(71) Applicant: AMBU A/S, Ballerup (DK)

(72) Inventor: Abbas AL ATRAKTCHI, Copenhagen S (DK)

(73) Assignee: AMBU A/S, Ballerup (DK)

(21) Appl. No.: 19/647,616

(22) Filed: Apr. 14, 2026

(30) Foreign Application Priority Data
Apr. 15, 2025 (EP) 25170551.3

(51) Int. Cl. A61B 10/04; A61B 1/00; A61B 1/015

(52) U.S. Cl. CPC A61B 10/04; A61B 1/00119; A61B 1/015

(57) ABSTRACT

A medical sampler (2) attachable to a suction connector (3) of an endoscope (4), to a sample container (6) and to a suction source (13) and operable in a sampling-mode and a bypass-mode. The medical sampler (2) includes a pinch valve (8) having a compressible tubing unit (10) and a pinching unit (12) configured to pinch the compressible tubing unit (10) in a first pinching state providing the sampling-mode and a second pinching state providing the bypass-mode.

### CROSS-REFERENCE TO RELATED APPLICATIONS

The present application claims the benefit of and priority from European Patent Application No. 25170551.3, filed Apr. 15, 2025; the disclosure of said application is incorporated by reference herein in its entirety.

### TECHNICAL FIELD

The present disclosure relates to a medical sampling device, in particular a sampler bridge attachable to a suction connector of an endoscope, to a sample container, and to a suction source, and to a system comprising the medical sampling device.

### BACKGROUND

Endoscopes, which include specialized instruments such as bronchoscopes, are used for visual examination and diagnosis of hollow organs and body cavities, as well as to assist in surgery, e.g. for a targeted tissue sampling. Endoscopes, both reusable and single-use, usually comprise a handle via which an operator can hold and control the endoscope, and an insertion cord comprising an insertion tube, an actively bendable bending section, and a distal tip unit. The insertion cord is configured to be inserted into the hollow organs and body cavities of a patient.

A bronchoalveolar lavage (BAL) is a typical procedure performed with a bronchoscope. BAL is used to collect a sample of fluid from the lower respiratory tract, specifically the bronchioles and alveoli, for diagnostic or research purposes. To perform this bronchoscopic procedure, a bronchoscope is guided into a specific region of the lung, a sterile saline solution is instilled into the lung segment, and the fluid is gently suctioned back, collecting cells, microorganisms, proteins and other components from the alveolar space.

The endoscopic procedure may involve suctioning fluids without sampling them. For example, body fluids may be suctioned to enable a camera of the endoscope to obtain usable images within the body cavity or to remove debris such as mucus or blood. Therefore, it is preferable to provide a valve configured to direct the fluid mixture suctioned through the endoscope to a sample container or to bypass the sample container and direct the fluid mixture to a waste container.

To reduce labor and complexity, it is preferred that one operator handles the endoscope, the medical sampling device, and the sample container. The medical sampling device should be as small as possible so that the system comprising the endoscope, the sampling device, and the sample container does not become unwieldy to handle by the operator. Additionally, the sampling device should minimize fluid pressure drop so that the vacuum provided by a vacuum source connected to the sampling device, and through the sampling device to the working channel valve, is fully used to suction the fluid mixture from the patient. Full use of the vacuum will reduce the duration of the endoscopic procedure by suctioning fluids more quickly than if less vacuum were used. Additionally, since the endoscope and the patient may move, it is desirable, when the tip of the endoscope is in the desired position, to withdraw the samples quickly before any movement dislodges the tip of the endoscope from the desired position. Even further, it is desirable to withdraw the samples quickly before any body fluids refill the space where the samples are to be taken. These requirements contravene since reduced size limits the space available to provide fluid channels, which should be as large as possible to reduce the fluid pressure drop.

### SUMMARY

The tasks and objectives of the present technology are to eliminate or at least to reduce the disadvantages of the related art. In particular, a medical sampling device, or sampler, shall be provided that reduces the pressure loss through the medical sampling device, has a simple design and can be easily assembled, and is small enough to be rigidly attached to the endoscope.

The present technology relates to a medical sampling device, also referred to as a medical sampler or sampler bridge, attachable to and between a suction connector of an endoscope, a sample container and a suction source. The medical sampler comprises a pinch valve including a pinching unit, a first conduit, a second conduit, a third conduit and a suction opening. The pinching unit is configured place the pinch valve in a sampling mode or a bypass mode. In the sampling-mode the first conduit and the second conduit are, concurrently, in an open configuration and the third conduit is in a closed configuration, whereby suction applied at the suction opening creates suction through the second conduit and the first conduit but not the third conduit. In the bypass-mode the first conduit and the second conduit are, concurrently, in a closed configuration and the third conduit is in an open configuration, whereby the suction applied at the suction opening creates suction through the third conduit but not through the second conduit and not through the first conduit.

The pinch valve provided within the medical sampling device advantageously offers a simple pinching solution, via which internal fluid flow can be suitably adjusted, in particular reliably prevented via a pinching engagement between the pinching unit and the conduits at desired positions, thereby realizing at least the sampling-mode and the bypass-mode. Advantageously, the pinching engagement leads to a reduced pressure loss relative to prior art rotary valves, at least due to the reduction or elimination of fluid connections, e.g. no need for separate sealings and no disconnection of flow channels, within the valve, so the risk of leaks is reduced or even eliminated. Such leaks not only reduce suction pressure, potentially extending the medical procedure, but also may allow escape of biomaterial that could increase infection risks.

Furthermore, by using the disclosed medical sampling device the pressure loss in the medical sampling device can be reduced, allowing consistent suction in the sampling-mode and the bypass-mode. This further enhances procedural efficiency and reduces patient discomfort.

The first conduit, the second conduit, and the third conduit may be integrally formed. In one example, a tube extends from the endoscope end to the sample container end of the pinch valve, the tube including the second conduit and the third conduit, the tube further comprising the suction opening in a wall of the tube, the suction opening being located between the second conduit and the third conduit. The tube and the first conduit may be integrally formed. The first conduit, the second conduit, and the third conduit may be integrally formed in a compressible tubing unit.

The first conduit, the second conduit, and the third conduit are compressible. A compressible conduit undergoes elastic deformation in response to an externally applied force and substantially returns to its undeformed shape upon removal of the force. Elastic deformation brings opposing wall portions of the conduit into contact to reduce a cross-sectional area of a lumen of the conduit and thus occlude fluid flow. Occlusion may be substantially complete or, preferably, complete. By substantially complete it is meant that any vacuum loss through the occluded conduit is de minimis.

The compressible conduit may be comprised or consist essentially of a thermoplastic elastomer. Example thermoplastic elastomers include styrenic block copolymers (e.g., SBS, SEBS), thermoplastic polyurethanes, thermoplastic polyolefin elastomers, thermoplastic vulcanizates, copolyester elastomers, and polyamide elastomers.

Elastic deformation may be provided by two pinching portions coming close together with the conduit therebetween. A pinching portion refers to any edge, surface, or geometric feature of a component configured to apply compressive force to the conduit to reduce its internal cross‑section. The pinching portions may be traversed by a common plane or be traversed by two parallel planes. Two opposed pinching portions form a pinch area corresponding in size to the lengths of the pinching portions and twice a wall thickness of the conduit. The pinch area is formed when the pinch portions bring opposing wall portions of the conduit into contact to occlude a cross-sectional area of the lumen.

In one embodiment, the pinch valve includes a drum housing, a flow connector body, and a drum rotatable within the drum housing to pinch the flow connector body and thereby alternate a state of the pinch valve between the sampling mode and the bypass mode. The flow connector body comprises a proximal inlet, a sampling tube (or first conduit) extending distally from the proximal inlet, a suction tube (or third conduit) extending distally from the proximal inlet, and an outlet tube extending at a non-zero angle from the suction tube. The drum and the drum housing cooperate to form three pinch areas including a sampling pinch area, a first bypass pinch area, and a second bypass pinch area. In the sampling mode the drum and the drum housing form the sampling pinch area, and in the bypass mode the drum and the drum housing form the first and the second bypass pinch areas.

In some embodiments, the pinch valve may comprise an actuation unit configured to switch the pinch valve between the bypass and the sampling mode. Preferably, the actuation unit is a mechanical actuation unit, in particular is or comprises a rotatable or displaceable actuation element or part or portion, e.g. a lever or switch or wheel part or portion. Alternatively, the actuation unit may be an electrical actuation unit, which e.g. comprise an electrical switch, a control unit and an electric motor rotating or translating the pinching unit. The actuation unit may be referred to as a “drum actuator.”

The pinch valve may comprise a tubing holder configured and provided to hold the compressible tubing unit.

The sampling-mode is a sample-collection-mode in which a specimen sample from the endoscope is collected in the sample container. The bypass-mode is a suctioning-mode in which the specimen sample is directly guided towards the suction source, thereby bypassing the sample container.

The pinch valve may comprise a compressible tubing unit and a pinching unit, the pinching unit being configured to pinch the compressible tubing unit providing the sampling-mode and the bypass-mode. The compressible tubing unit may comprise the first, second and third conduits.

Preferably, the medical sampling device comprises a housing, in which the pinch valve is disposed.

The pinching unit of the pinch valve may be integrally formed, integrally joined, or may comprise a plurality of pinching parts.

The pinch valve may comprise a tubing holder configured and provided to hold the compressible tubing unit. The compressible tubing unit may be an integral part or may comprise a plurality of tubes.

The compressible tubing unit may comprise a first fluid connection having a first endoscope end and a first sample container end, and a second fluid connection having a second endoscope end, a second sample container end and a suction source end. The second fluid connection may have an endoscope portion, a sample container portion and a suction source portion, each extending from a common connection point, i.e. the second fluid connection may essentially be formed in a T-shape.

The pinching unit may be configured to pinch the compressible tubing unit in a first pinching state providing the sampling-mode such that fluid flow or suctioning is enabled from the first endoscope end to the first sample container end, via the sample container to the second sample container end and from the second sample container end to the suction source end. In particular, the pinching unit may be configured to pinch the compressible tubing unit in the first pinching state providing the sampling-mode such that fluid flow is prevented between the second endoscope end and the suction source end. Further, the pinching unit may be configured to pinch the compressible tubing unit in the first pinching state providing the sampling-mode at a third position on the second fluid connection, the third position may be a position upstream of the connection point on the second fluid connection, thereby preventing fluid flow between the second endoscope end and the second sample container end.

The pinching unit may be configured to pinch the compressible tubing unit in a second pinching state providing the bypass-mode such that fluid flow or suctioning is enabled between the second endoscope end and the suction source end. In particular, the pinching unit may be configured to pinch the compressible tubing unit in the second pinching state providing the bypass-mode at a first position on the first fluid connection, the first position may be an arbitrary position on the first fluid connection, thereby preventing fluid flow via the first fluid connection. Further, the pinching unit may be configured to pinch the compressible tubing unit in the second pinching state providing the bypass-mode at a second position on the second fluid connection, the second position may be a position downstream of the connection point on the second fluid connection, thereby enabling fluid flow between the second endoscope end and the suction source end and preventing fluid flow towards and from the second sample container end.

According to an embodiment, the pinching unit may be a rotating part having an axis of rotation parallel or substantially parallel to the longitudinal axis.

Further, according to an embodiment, the pinching unit may be a rotating part having an axis of rotation arranged essentially perpendicularly to the longitudinal axis.

Furthermore, according to an embodiment, the pinching unit may be formed as the rotating part and may comprise a first engagement end or portion (e.g. a wide clamping portion) configured and provided to pinch the first fluid connection and the second fluid connection, and a second engagement end or portion (e.g. a narrow clamping portion) configured and provided to pinch only the second fluid connection (i.e. not the first fluid connection).

Preferably, the first engagement end or portion has an engagement length greater than an engagement length of the second engagement end or portion.

Furthermore, according to an embodiment, the first engagement end or portion may be arranged essentially diametrically opposed to the second engagement end or portion.

According to an embodiment, the pinching unit formed as the rotating part, in the first pinching state providing the sampling-mode, may be rotated in(to) a first position in which the second engagement end pinches the second fluid connection.

Furthermore, the pinching unit formed as the rotating part in the second pinching state providing the bypass-mode may be rotated in a second position in which the first engagement end pinches the first fluid connection and the second fluid connection.

The medical sampling device may have a locking or fixing mechanism, in particular a snap-in mechanism, configured and provided to lock or fixate the pinching unit in the first pinching state and/or in the second pinching state.

The locking or fixing mechanism, in particular the snap-in mechanism, may be formed by a protruding (elastic or spring) portion, preferably of the pinching unit, in particular of the rotating part, engaging in a recessed portion, preferably of a stationary part like the housing or the tubing holder.

According to an aspect of the technology, an additional locking mechanism is not necessary, if the internal friction between the rotating part and the stationary part is set to prevent elastic restoring displacements of the pinched tubing unit. Moreover, a separate fixing mechanism is also not necessary according to another aspect of the technology, when electrical components are used to rotate the rotating part, since a motor inherently has a stopping effect. This is due to the fact that when a power to the motor is cut off, it naturally ceases movement due to an internal resistance, friction, or the way the motor is designed to stop. Therefore, the built-in stopping mechanism of the motor may eliminate the need for an additional snap mechanism to halt its motion.

It has been found that silicone, TPU and rubber are preferable materials for medical pinched tubes due to their flexibility, durability and biocompatibility. Silicone is highly resistant to temperature extremes, while TPU offers excellent elasticity and chemical resistance. Rubber provides resilience and reliable sealing.

The present technology further relates to a system comprising the medical sampling device according to aforementioned embodiments and examples, and a sample container, preferably also an endoscope.

The system may further comprise a suction source (device) that is connectable directly or indirectly, for example through a tube or hose, to the suction source connection interface.

The suction source may be a vacuum pump or the like.

### BRIEF DESCRIPTION OF THE DRAWINGS

The above-mentioned embodiments, features and advantages thereof will be further elucidated by the following illustrative detailed description of the present technology with reference to the appended drawings. The drawings illustrate embodiments, variations and examples of the present technology to facilitate the understanding of a person of ordinary skill in the art and are not to be construed as limiting the scope of the claims.

FIG. 1 shows an exploded perspective view of a system comprising a medical sampler, an endoscope and a sample container.

FIG. 2 shows a perspective view of the medical sampling device according to a first embodiment.

FIG. 3 shows an exploded view of the medical sampling device according to the first embodiment.

FIG. 4 shows a perspective view of a sample container connection interface of the medical sampling device according to the first embodiment.

FIG. 5 shows a perspective view of an endoscope connection interface of the medical sampling device according to the first embodiment.

FIG. 6 shows a perspective view of a housing accommodating a pinch valve of the medical sampling device according to the first embodiment.

FIG. 7 shows a perspective view of a tubing holder of the medical sampling device according to the first embodiment.

FIG. 8 shows a perspective view of a pinching unit of the medical sampling device according to the first embodiment.

FIG. 9 shows a perspective view of a compressible tubing unit of the medical sampling device according to the first embodiment.

FIG. 10 shows a perspective view of a pinch valve of the medical sampling device according to the first embodiment in a sampling-mode.

FIG. 11 shows a perspective view of the pinch valve of the medical sampling device according to the first embodiment in a bypass-mode.

FIG. 12 shows an exploded cross-sectional view of the medical sampling device according to the first embodiment with illustrated pinching positions.

FIG. 13 shows a perspective view of the compressible tubing unit of the medical sampling device according to the first embodiment with illustrated pinching positions in the sampling mode.

FIG. 14 shows a perspective view of the compressible tubing unit of the medical sampling device according to the first embodiment with illustrated pinching positions in the by-pass mode.

FIGS. 15A and 15B are schematic views showing an endoscope-side view of the endoscope end of the pinch valve illustrating pinching positions.

FIGS. 16A and 16B are schematic views showing an endoscope-side view of the sample container end of the pinch valve illustrating pinching positions.

FIG. 17 is a schematic view depicting a perimeter of an aperture of the pinch valve.

FIG. 18 shows a perspective view of a medical sampling device according to a second embodiment.

FIG. 19 shows an exploded perspective view of the medical sampling device according to the second embodiment.

FIG. 20 shows a schematic illustration of a pinch valve prototype of a medical sampling device according to a third embodiment.

FIG. 21 shows a schematic illustration of a pinch valve prototype of a medical sampling device according to a fourth embodiment in a sampling-mode.

FIG. 22 corresponds to FIG. 21 but with the pinch valve in bypass-mode.

FIG. 23 shows a perspective view of the medical sampling device according to the fourth embodiment.

FIG. 24 shows a sectional view of a snap-in mechanism.

In the following description, the same reference numbers refer to the same elements throughout the figures.

### DETAILED DESCRIPTION

Unless expressly specified otherwise, the following terms shall have the meanings specified in the section titled “Interpretation and Terminology”: about, approximately, comprising, consisting, consisting substantially of, a/an, or, between or “in the range,” based on, coupled/connected/communicatively coupled, determining, integral, distal, videoscope, endoscope, plurality, and positioning interface.

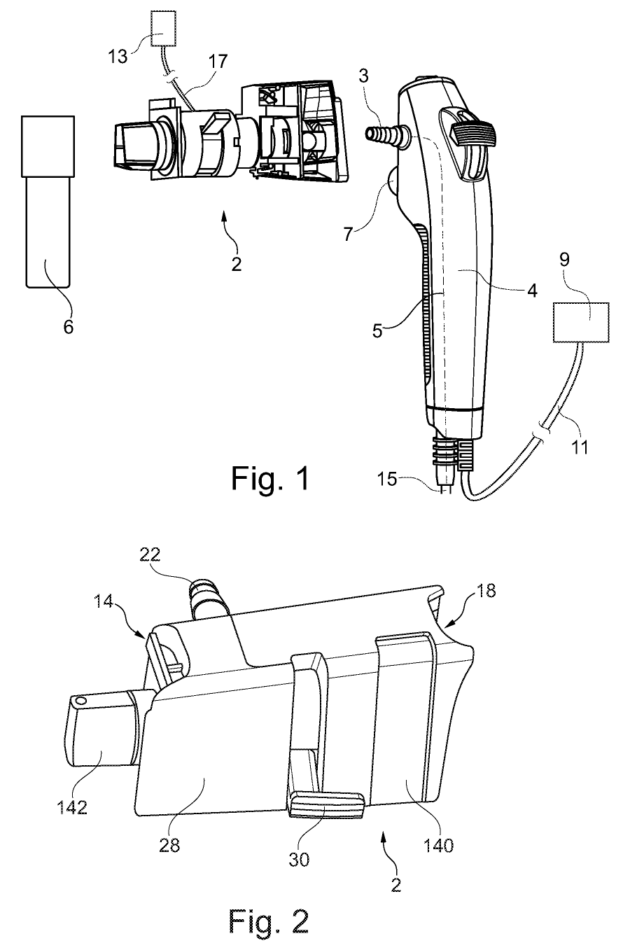

FIG. 1 is an exploded perspective view of a sampling system or set comprising a medical sampling device 2, an endoscope 4 and a sample container 6. The medical sampling device 2 is connectable to the sample container 6 and the endoscope 4. The endoscope 4 comprises an insertion cord 15 (only shown in part) having a proximal end connected to a handle of the endoscope and a distal end insertable in a body cavity of a patient. A working channel 5 runs through the insertion cord to an opening at the distal end of the insertion cord. At the other end the working channel 5 may continue inside the handle as illustrated and end at a suction connector 3. The suction connector 3 may be directly or indirectly connected to a suction source 13. In the illustrated example the suction source 13 is fluidly connected via a hose 17 to the medical sampling device 2, which in turn is ready for attachment to the suction connector 3 of the endoscope 4 for fluidly connecting to the working channel 5 of the endoscope 4. Suction through the endoscope may be controlled by a valve (not shown) operated by a suction button 7. The sample container 6 may in turn be connected to the medical sampling device 2. The endoscope distal tip may further include a light source and a camera connectable to a video processing apparatus and a monitor 9 via a cable 11 allowing the operator to monitor the interior of the body cavity and activities performed. The medical sampling device 2 may be a medical sampler according to any of the embodiments, variations and examples thereof discussed herein.

The endoscope may be a flexible endoscope or a rigid endoscope. The endoscope may be a reusable or a single-use endoscope. Preferably, the endoscope is a single-use endoscope. Especially preferred also the medical sampling device and the sample container are single use devices or components. Further, the endoscope is preferably a bronchoscope, however may alternatively also be a gastroscope, a colonoscope, a laparoscope, an arthroscope, a cystoscope, a hysteroscope, etc.

Since the medical sampling device is attachable to the endoscope, which is preferably a bronchoscope, and to the sample container, it is easy to perform a bronchoalveolar lavage during a bronchoscopic procedure, in particular when the medical sampling device is also attachable to a suction source enabling fluid flow and suctioning both in the bypass-mode and the sampling-mode.

Furthermore, since the sample container is attachable and detachable from the medical sampling device, the sample container can be exchanged during usage after switching from the sampling-mode to the bypass-mode. An exchangeable sample container in a set or system comprising at least the medical sampling device and the sample container, preferably also the endoscope, offers the advantage that a desired probe volume is not restricted by a volume of the sample container. The aforementioned modularity may improve workflow efficiency, since multiple samples can be collected sequentially without interrupting the endoscopic, in particular bronchoscopic, procedure. Additionally, an exchangeable sample container enables precise sample handling and storage, reducing the risk of contamination or degradation before laboratory analysis. In research and diagnostic settings, this feature may support higher throughput and consistency, making the system or set with the disclosed medical sampling device more versatile and cost-effective.

The bypass-mode and the sampling-mode in such a system or set enable an appropriate sample collection in the sample container in the sampling-mode, without interrupting suction when the sample container is full, since a user can then easily and reliably switch to the bypass-mode. This offers advantages in both clinical and procedural efficiency. By maintaining continuous suctioning, uninterrupted operation is ensured. The seamless transition between containers enhances procedural workflow, eliminating the need for system pauses or manual interventions, which can prolong the examination or treatment. Additionally, the uninterrupted suction reduces patient discomfort by minimizing procedural delays and ensuring a smoother, more efficient experience. This advantageously enhances user control, improves sample integrity and contributes to a more patient-friendly and hygienic endoscopic procedure.

The medical sampling device may comprise a sample container connection interface adapted/ designed for connection to the sample container. In particular, the medical sampling device may comprise a sample container attachment element or portion, on which the sample container connection interface is formed. The sample container connection interface is a direct or immediate interface or contact point, where the sample container is attached to the medical sampling device. The sample container connection interface may be provided on a separate sample container attachment element or part. Alternatively, the sample container connection interface may be provided on a portion of another part or component of the medical sampling device, i.e. may be provided on a sample container attachment portion of another part or component of the medical sampling device.

The medical sampling device may comprise an endoscope connection interface adapted/ designed for connection to the endoscope. In particular, the medical sampling device may comprise an endoscope attachment element or portion, on which the endoscope connection interface is formed. The endoscope connection interface is a direct or immediate interface or contact point, where the endoscope is attached to the medical sampling device. The endoscope connection interface may be provided on a separate endoscope attachment element or part. Alternatively, the endoscope connection interface may be provided on a portion of another part or component of the medical sampling device, i.e. may be provided on an endoscope attachment portion of another part or component of the medical sampling device.

The medical sampling device may comprise a suction source connection interface adapted for connection to a suction source. In particular, the medical sampling device may comprise a suction source attachment element or portion, on which the suction source connection interface is formed. The suction source connection interface is a direct or immediate interface or contact point, where a suction source or a tube or hose leading to the suction source is attached. The suction source connection interface may be provided on a separate suction source attachment element or part. Alternatively, the suction source connection interface may be provided on a portion of another part or component of the medical sampling device, i.e. may be provided on a suction source attachment portion of another part or component of the medical sampling device.

The medical sampling device may comprise a first axial end on which the endoscope is attachable and a second axial end (opposite the first axial end) on which the sample container is attachable. Of course, the endoscope is preferably also detachable from the first axial end, and the sample container is preferably also detachable from the second axial end. The endoscope and the sample container are not necessarily arranged opposite to each other on opposing axial ends according to the present technology. For example, it is also possible to connect the endoscope or the sample container substantially perpendicularly to each other.

According to a preferred embodiment, the endoscope connection interface is provided on the first axial end, the sample container connection interface is provided on the second axial end, and a longitudinal axis is formed between the first axial end and the second axial end.

The suction source connection interface may be oriented at an angle with respect to the longitudinal axis, preferably at a right angle.

FIG. 2 is a perspective view of the medical sampling device 2 according to the first embodiment. The medical sampling device 2 is in a fully assembled state and therefore compact and aesthetical appealing. In the illustrated assembled state, a housing 28, a sample container connection interface 14, an endoscope connection interface 18, a suction source connection interface 22, a mechanical actuation unit 30, a locking key 140 and a cover 142 for the sample container connection interface 14 are visible. When engaged and fully assembled (i.e. when the endoscope 4 is connected to the endoscope connection interface 18), the locking key 140 locks the medical sampling device 2 to the endoscope 4 in a fixed position. The mechanical actuation unit 30 comprises an L-shaped fluted section, which is adapted to be easily touched/ gripped by the user in an ergonomic way. Further, the suction source connection interface 22 is barbed in order to securely connect to a hose. As can be seen, the cover 142 fully sheathes fluid openings of the sample container interface 14, to safeguard them from external contamination and other possible unintended influences.

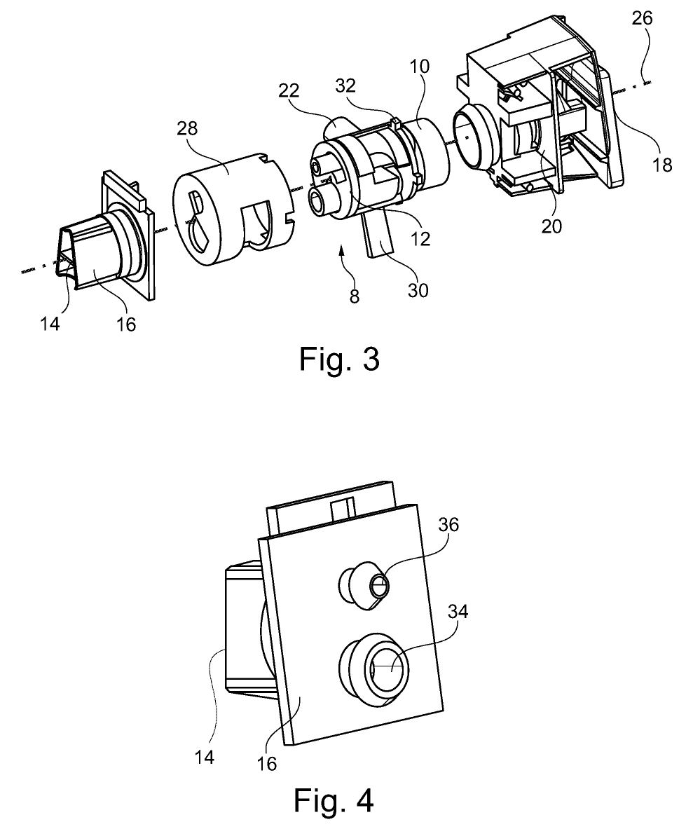

FIG. 3 is an exploded view of the medical sampling device 2 according to the first embodiment. The medical sampling device 2 comprises the sample container connection interface 14 provided on a sample container attachment element 16, the endoscope connection interface 18 provided on an endoscope attachment element 20, the housing 28 and a pinch valve 8, which is accommodated in the housing 28. The pinch valve 8 comprises the suction source interface 22. The sample container attachment element 16, the housing 28, the pinch valve 8 and the endoscope attachment element 20 are all arranged along a longitudinal axis 26.

The pinch valve 8 comprises a compressible tubing unit 10, which is pinchable by a pinching unit 12, and a tubing holder 32, which is configured and provided to hold the compressible tubing unit 10. Further, the pinching unit 12 has the mechanical actuation unit 30, which is formed as a lever.

A sampling-mode and a bypass-mode can be achieved by rotating the pinching unit 12, in particular the mechanical actuation unit 30 in form of the lever, around a rotational axis, which substantially corresponds to the longitudinal axis 26 in the first embodiment of the medical sampling device 2. A rotation of the mechanical actuation unit 30, i.e. of the pinching unit 12, leads to a rotational movement of the pinching unit 12 relative to the tubing holder 32 and the housing 28. By rotating the pinching unit 12 with respect to the housing 28 and the tubing holder 32, the compressible tubing unit 10 is pinched in predetermined positions. The pinching of the compressible tubing unit 10 in the predetermined positions defines the respective modes, namely the sampling-mode and the bypass-mode. Preferably, the pinching unit 12, in particular the mechanical actuation unit 30, is rotated or rotatable around 90° in order to switch between the sampling-mode and the bypass-mode. The medical sampling device 2 preferably allows just this one internal degree of freedom, which is the rotation of the pinching unit 12 around the longitudinal axis 26. Pinching may be effected for example by squeezing or bending the tube to close the inner opening and cut the flow.

Further, the components or parts of the medical sampling device 2 can be divided into a stationary group and a rotatable group. The stationary group comprises the tubing holder 32, the compressible tubing unit 10, the housing 28, the sample container attachment element 16 and the endoscope attachment element 20. The rotatable group comprises the pinching unit 12. The system borders of the internal degrees of freedom do not include external degrees of freedom, such as movements of the entire medical sampling device 2 or the entire set comprising the medical sampling device 2, the endoscope 4 and the sample container 6, e.g. during use, assembly, disassembly, etc. and movements of connection elements, which occur when the endoscope 4 is connected or disconnected, or when the sample container 6 is connected or disconnected, for example.

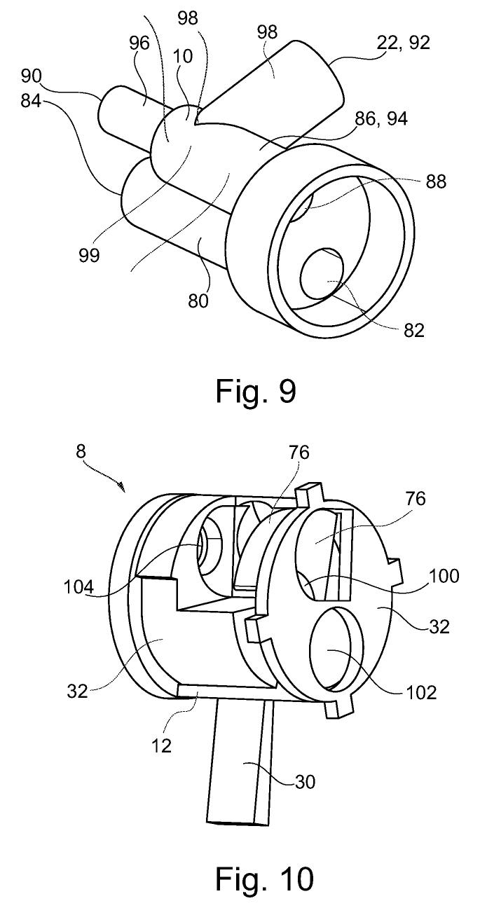

FIG. 4 shows a perspective view of the sample container attachment element 16 having the sample container connection interface 14 according to the medical sampling device 2 of the first embodiment of the present technology. The sample container attachment element 16 comprises a first tubing connection 34 and a second tubing connection 36, which are configured to connect to the compressible tubing unit 10 (see FIG. 9). The sample container attachment element 16, in particular the sample container connection interface 14 is further configured to connect to the sample container 6 in a safe manner.

Preferably, the connections of the first tubing connection 34 and the second tubing connection 36 are achieved by barbed fittings or connectors, which enable a fluid connection between the sample container 6 and the compressible tubing unit 10 while additionally providing fluid-impermeable sealings, in particular when the compressible tubing unit 10 is connected to the first and second tubing connections 34, 36 of the sample container attachment element 16, for a safe separation of the liquid from the environment and vice versa.

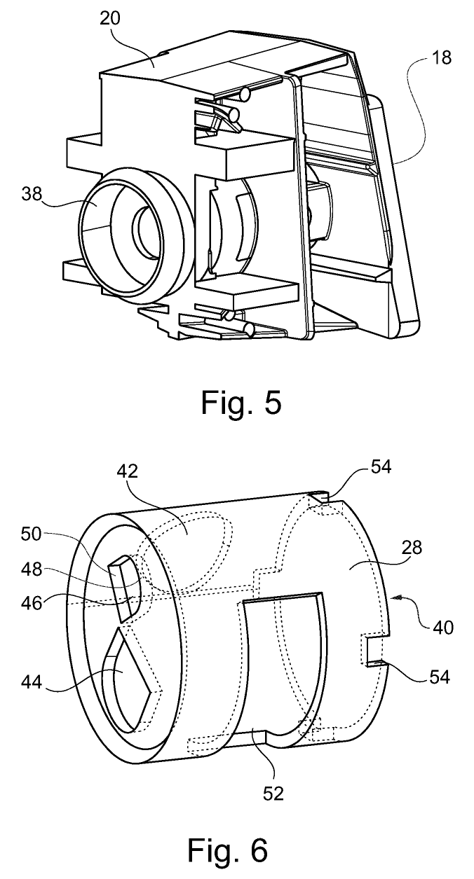

FIG. 5 shows a perspective view of the endoscope attachment element 20 having the endoscope connection interface 18 according to the medical sampling device 2 of the first embodiment of the present technology. The endoscope attachment element 20 comprises a tubing connection 38, which is configured to connect to the compressible tubing unit 10.

Preferably, the connection of the tubing connection 38 is achieved by a barbed fitting or connector, which enables a fluid connection between the endoscope 4 and the compressible tubing unit 10, in particular between the endoscope attachment element 20 and the compressible tubing unit 10, while additionally providing fluid-impermeable sealings, in particular when the compressible tubing unit 10 is connected to the tubing connection 38, for a safe separation of the liquid from the environment and vice versa. Alternatively, the compressible tubing unit could be configured for attachment directly to the endoscope, such as directly to the suction connector 3.

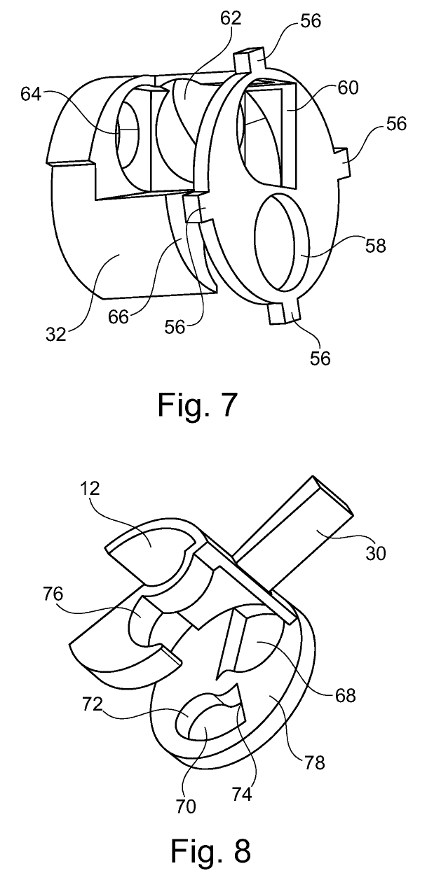

FIG. 6 shows a perspective view of a housing 28 according to the first embodiment of the medical sampling device 2. The housing 28 comprises a housing endoscope opening 40, a housing suction source opening 42, a first housing sample container opening 44, a second housing sample container opening 46, an actuation unit opening 52 and preferably a rotational lock to hinder mutual rotation of the housing 28 in relation to the tubing holder 32, here in the form of four tubing holder recesses 54, as will be described in relation to FIG. 7 below.

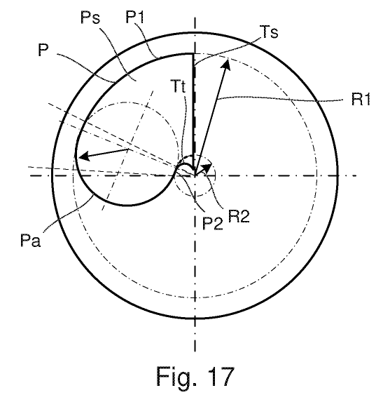

The perimeter of the first housing sample container opening 44 and of the second housing sample container opening 46 each comprise a curved portion 48 and a straight portion 50. The straight portion 50 forms a hold or support for the tube when pinching. The first housing sample container opening 44 may be bigger than the second housing sample container opening 46. As shown, the curved portion comprises a semicircular arc. A slot portion may extend from the semicircular arc to the straight portion 50. The slot portion may be defined by a first boundary segment and a second boundary segment, the first boundary segment having a constant curvature having a first radius, and the second boundary segment having a constant curvature having a second radius smaller than the first radius. The slot portion is described in detail with reference to FIG. 17. The first housing sample container opening 44 and of the second housing sample container opening 46 in the present example comprise a perimeter with a D-shape. The perimeter may also comprise the shape shown and described with reference to FIG. 17. The straight portion 50 may be referred to as a pinching portion.

The actuation unit opening 52 preferably is a rectangular cutout in a cylindrical wall of the housing 28, further preferably configured to allow substantially a 90° rotation (angle) of the mechanical actuation unit 30, in an assembled state of the medical sampling device 2.

FIG. 7 shows a perspective view of a tubing holder 32 according to the first embodiment of the medical sampling device 2. The tubing holder 32 comprises preferably four tubing holder protrusions 56, which are configured as mating protrusions corresponding to the tubing holder recesses 54 of the housing 28 to form a rotational lock between the housing 28 and the tubing holder 32.

Further, the tubing holder 32 comprises a first tubing holder endoscope opening 58, a second tubing holder endoscope opening 60, a tubing holder suction source opening 62, a first tubing holder sample container opening (not shown), a second tubing holder sample container opening 64 and a clamping unit recess 66. The perimeter of the second tubing holder endoscope opening 60 comprises a curved portion and a straight portion, similar to the first housing sample container opening 44. As shown, the curved portion comprises a semicircular arc. A slot portion may extend from the semicircular arc to the straight portion. The slot portion may be defined by a first boundary segment and a second boundary segment, the first boundary segment having a constant curvature having a first radius, and the second boundary segment having a constant curvature having a second radius smaller than the first radius. The slot portion is described in detail with reference to FIG. 17. The second tubing holder endoscope opening 60 in the present example comprises a perimeter with a D-shape. The perimeter may also comprise the shape shown and described with reference to FIG. 17. The straight portion may be referred to as a pinching portion.

FIG. 8 shows a perspective view of the pinching unit 12 according to the first embodiment of the medical sampling device. The pinching unit 12 comprises a first clamping portion 68, a second clamping portion 70 and a clamping claw 76. The first clamping portion 68 and the second clamping portion 70 each comprise a curved portion 72 and a straight portion 74.

The clamping claw 76 is configured to be accommodated in the clamping unit recess 66 as shown in FIG. 7 when assembled. Moreover, the pinching unit 12 comprises the manual actuation unit 30, which has the form of a lever, and which may be mounted to the pinching unit 12 after the parts are assembled. The first clamping portion 68 and the second clamping portion 70 may comprise, as shown, a perimeter described with reference to FIG. 17, comprising a pointed or tapered end.

FIG. 9 shows a perspective view of a compressible tubing unit 10 of the medical sampling device 2. The compressible tubing unit 10 may be integrally formed or joined and comprises a first fluid connection, or conduit, 80 having a first endoscope end 82 and a first sample container end 84, and a second fluid connection, or tube, 86 having a second endoscope end 88, a second sample container end 90 and a suction source end 92. Further, the second fluid connection 86 of the compressible tubing unit 10 comprises an endoscope portion 94, a sample container portion 96 and a suction source portion 98, which combined preferably form a T-shape. These three portions come together at, or in other words extend from a common point 99. A suction opening 98a is provided in the tube 86 and the suction source portion 98 extends from the suction opening 98a.

In the present embodiment, the tube 86 comprises a second conduit 86a and third conduit 86b, the second and third conduit being separated by the suction opening 98a.

In a variation of the present embodiment, the first conduit 80 and the tube 86 are not integrally formed or joined. In an example, the tube 86 comprises a second conduit 86a and third conduit 86b, the second and third conduit being separated by the suction opening 98a.

When the medical sampling device 2 is in an assembled state comprising the compressible tubing unit 10 (FIG. 9), the housing 28 (FIG. 6), the tubing holder 32 (FIG. 7) and the pinching unit 12 (FIG. 8), the first fluid connection 80 (or sample container inlet channel) is configured to fluidly connect the endoscope 4 with the sample container 6 and the second fluid connection is configured to fluidly connect the endoscope 4, the sample container 6 and a suction source (not shown). Thereby, the first fluid connection 80 passes through the housing endoscope opening 40, the first tubing holder endoscope opening 58, the first tubing holder sample container opening (not shown), the first clamping portion 68 and the first housing sample container opening 44. Furthermore, the second fluid connection 86 passes through the housing endoscope opening 40, the second tubing holder endoscope opening 60, the clamping claw 76, suction source opening 62, the second tubing holder sample container opening 64, the second clamping portion 70 and the second housing sample container opening 46.

FIG. 10 shows a perspective view of part of the pinch valve 8 in a sampling-mode. The compressible tubing unit 10 is omitted as well as the housing 28 for better showing the pinching functionality. As can be seen, the passage 100 is closed by the clamping claw 76, which pinches the compressible tubing unit 10 in a third pinching position within the endoscope portion 94. Consequently, the second fluid connection 86 is fully closed within the endoscope portion 94, just allowing fluid flow through the first fluid connection 80, the sample container portion 96, which lies within the open passage 104, and the suction source portion 98. In other words, no fluid can flow from the endoscope 6, directly to the suction source (not shown), while bypassing the sample container 6.

The closed passage 100 is formed, when a corresponding curved portion of the pinching unit 12 moves closer to the corresponding curved portion of the tubing holder 32 by the rotation of the pinching unit 12, thereby clamping the compressible tubing unit 10. Due to the omitted compressible tubing unit 10, it can be seen that the opening left in the closed passage 100 is oval shaped, or rather pointed oval or almond-shaped.

When the pinch valve 8 is in an assembled state, then a combined first and second position portion 78 of the pinching unit 12 lies outside the tubing holder 32. In other words, an inner surface of the combined first and second position portion 78 abuts against an outer surface of the tubing holder 32.

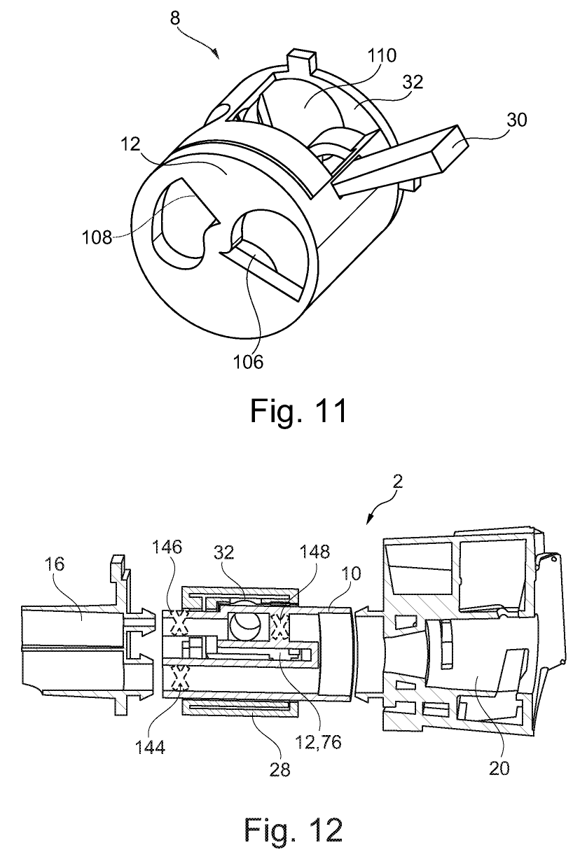

FIG. 11 shows a perspective view of part of the pinch valve 8 in a bypass-mode. The compressible tubing unit 10 as well as the housing 28 is omitted for better showing the pinching functionality. As can be seen, a passage 106 is closed by the first clamping portion 68, which pinches the compressible tubing unit 10 in a first pinching position on the first fluid connection 80. Consequently, the first fluid connection 80 is fully closed. Further, a passage 108 (concealed) is closed by the second clamping portion 70, which pinches the compressible tubing unit 10 in the sample container portion 96. Therefore, no fluid can flow from the sample container 6 to the suction source (not shown). By pinching the compressible tubing unit 10 in the described first and second pinching positions, fluid can just flow directly from the endoscope 4 to the suction source (not shown), through open passage 110, bypassing the sample container 6 completely. In other words, no fluid can flow from the endoscope 6 directly to the sample container 6.

The closed passage 106 is formed by one corresponding straight portion of the pinching unit 12 moving closer to the corresponding curved portion of the tubing holder 32, via the rotation of the pinching unit 12, thereby clamping the compressible tubing unit 10. Due to the omitted compressible tubing unit 10, it can be seen that the closed passage 106 has the shape of a circular segment.

The closed passage 108 is formed, when the corresponding straight portion of the pinching unit 12 moves closer to the corresponding curved portion of the tubing holder 32, via the rotation of the pinching unit 12, thereby clamping the compressible tubing unit 10. Due to the omitted compressible tubing unit 10, it can be seen that the closed passage 108 has the shape of a circular segment.

When the pinch valve 8 is in an assembled state, a combined first and second position portion 78 of the pinching unit 12 lies outside the tubing holder 32. In other words, an inner surface of the combined first and second position portion 78 abuts against an outer surface of the tubing holder 32.

FIG. 12 is an exploded cross sectional view of the medical sampling device 2 according to the first embodiment with illustrated pinching positions 144, 146, 148. Visible are the sample container attachment element 16, the endoscope attachment element 20, the housing 28, the pinching unit 12 with the clamping claw 76, the compressible tubing unit 10 and the tubing holder 32 sandwiched in-between in an assembled state. Further, FIG. 12 illustrates three X-shaped crosses, which are drawn at the pinching positions 144, 146, 148. It is mentioned, that the functionality of the medical sampling device 2 is not influenced, if the pinching positions 144, 146, 148 are slightly moved/ shifted. When the by-pass mode is used, the compressible tubing unit 10 is pinched/ clamped in the first pinching position 144 and the second pinching position 146. When the sampling mode is used, the compressible tubing unit 10 is pinched/ clamped in the third pinching position 148. Correspondingly, the first pinching position 144 and the second pinching position 146 are open in the sampling mode and respectively the third pinching position 148 is open in the by-pass mode. This leads in each case to just one possible fluid flow from the endoscope 4 to the suction source 13.

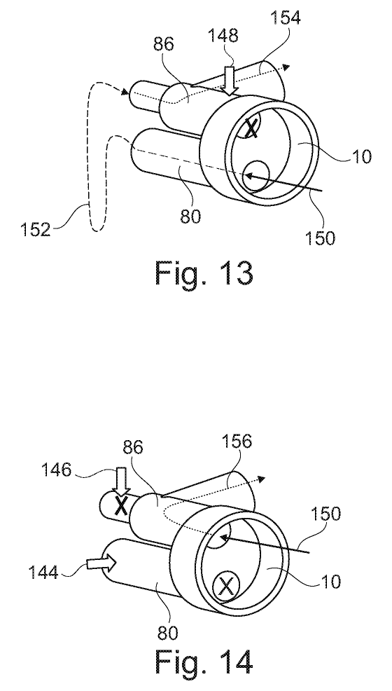

FIG. 13 is a perspective view of the compressible tubing unit 10 of the medical sampling device 2 according to the first embodiment with the illustrated pinching position 148 in the sampling mode. The compressible tubing unit 10 comprises the first fluid connection 80 and the second fluid connection 86. The black X-shaped cross indicate a corresponding closed fluid flow path. In the shown sampling mode, the fluid flows along a fluid way 150 through the first fluid connection 80, then enters and exits the sample container 6 (see FIG. 1) along a fluid way 152 and finally flows towards the suction source 13 (see FIG. 1) along a fluid way 154.

FIG. 14 shows a perspective view of the compressible tubing unit 10 of the medical sampling device according to the first embodiment with illustrated pinching positions 144, 146 in the by-pass mode. The compressible tubing unit 10 comprises the first fluid connection 80 and the second fluid connection 86. The black X-shaped crosses indicate corresponding closed fluid flow paths. In the shown by-pass mode, the fluid flows along the fluid way 150 through the second fluid connection 86 and then flows towards the suction source 13 (FIG. 1) along a fluid way 156.

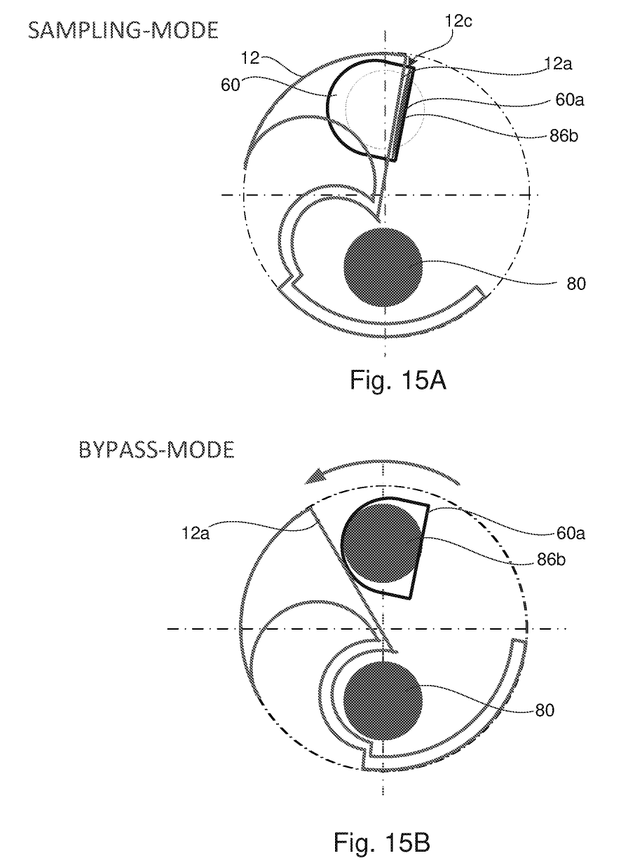

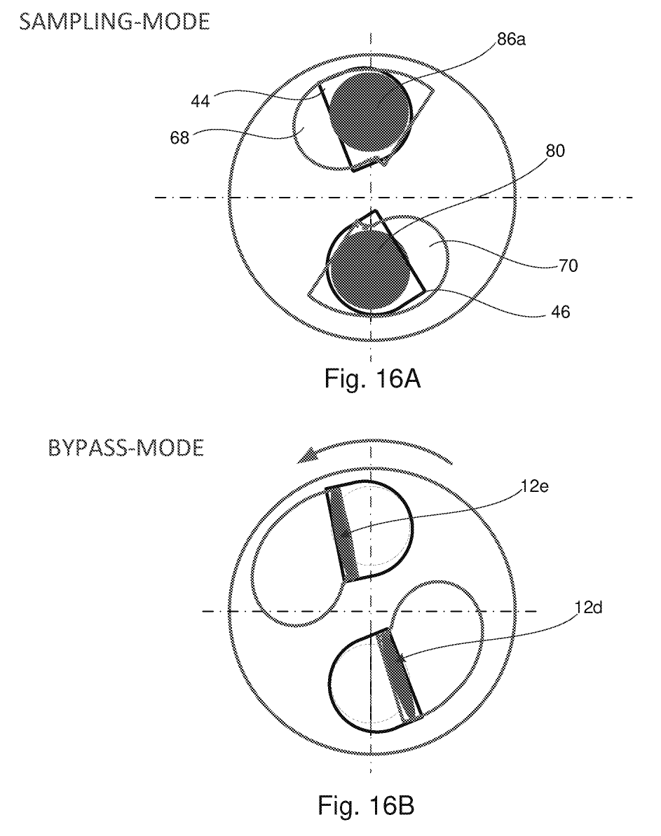

FIGS. 15A and 15B are schematic views showing an endoscope-side view of the endoscope end of the pinch valve illustrating pinching positions. FIGS. 16A and 16B are schematic views showing an endoscope-side view of the sample container end of the pinch valve illustrating pinching positions. FIG. 15A depicts the pinch valve in the sampling mode, and FIG. 15B depicts the pinch valve in the bypass mode. In FIG. 15a two opposed pinching portions 12a and 60a form a third pinch area 12c corresponding in size to the lengths of the pinching portions and twice a wall thickness of the third conduit 86b. The pinch area is formed when the pinch portions bring opposing wall portions of the third conduit into contact to occlude a cross-sectional area of the lumen of the third conduit. The first conduit 80 remains unpinched.

FIG. 16A depicts the pinch valve in the sampling mode, and FIG. 16B depicts the pinch valve in the bypass mode. In FIG. 16a two opposed pinching portions form a first pinch area 12d corresponding in size to the lengths of the pinching portions and twice a wall thickness of the first conduit 80. The pinch area is formed when the pinch portions bring opposing wall portions of the first conduit into contact to occlude a cross-sectional area of the lumen of the first conduit. Additionally, two opposed pinching portions form a second pinch area 12e corresponding in size to the lengths of the pinching portions and twice a wall thickness of the second conduit 86a. The pinch area is formed when the pinch portions bring opposing wall portions of the second conduit into contact to occlude a cross-sectional area of the lumen of the second conduit.

FIG. 17 is a schematic view depicting a perimeter of an aperture of the pinch valve. As previously stated, the medical sampler comprises the pinching unit, which comprises a housing extending from the endoscope end to the sample container end and comprising a longitudinal axis extending from the endoscope end to the sample container end; and a drum rotatable in the housing to switch the pinch valve between the bypass-mode and the sampling-mode. The housing comprises a first conduit opening 44 and a second conduit opening 46 (see e.g. FIG. 6), the first conduit 80 traversing the first conduit opening and the second conduit 86a traversing the second conduit opening. In one example shown in FIG. 17 the first conduit opening and the second conduit opening each comprises a perimeter P comprising: (i) a first end portion defined by a semicircular arc; (ii) a slot portion Ps extending from the semicircular arc and defined by a first boundary segment P1 and a second boundary segment P2, the first boundary segment having a constant curvature having a first radius R1, and the second boundary segment having a constant curvature having a second radius R2 smaller than the first radius; and (iii) a terminal portion T comprising a straight portion Ts and a transition portion Tt connecting the straight (pinching) portion with the second boundary segment, the transition portion transitioning the second boundary segment, in a continuous manner, from the second radius to a smaller radius culminating in a pointed or tapered end. The terminal portion allows a length of the cross-section of the conduit to increase as the conduit is compressed. The perimeter is configured to minimize a gap between the respective conduit and the perimeter so that the pinch valve can be made as small as possible.

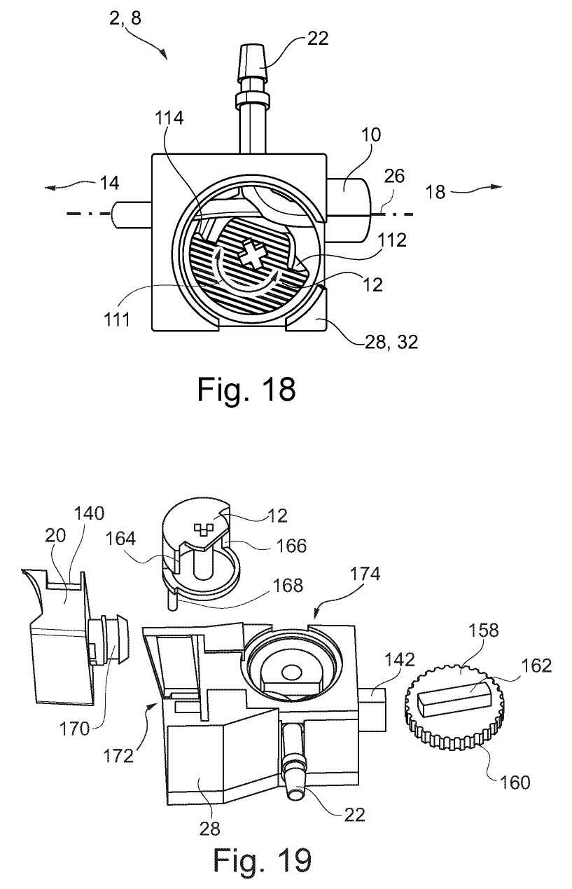

FIG. 18 shows a perspective view of the medical sampling device 2 according to a second embodiment, and FIG. 19 shows an exploded view of a variant of this embodiment. The functionality of the medical sampling device 2 is similar to the first embodiment, but in this second embodiment pinching is provided by pressing the compressible tubing against a back wall as will be discussed below. The rotational axis of the pinching unit 12 is perpendicular or substantially perpendicular to the longitudinal axis 26 and is indicated by the arrow 111. The rotation axis of the pinching unit 12 may be off-set relative to the axis 26 as shown, which has the advantage that assembly is facilitated. This embodiment comprises only few parts and assembly is simple, which makes it suitable for single-use medical devices where cost of manufacturing is important.

In the second embodiment, the medical sampling device 2 comprises a sample container connection interface 14, an endoscope connection interface 18, a suction source interface 22, a longitudinal axis 26, a pinch valve 8, wherein the pinch valve 8 comprises a compressible tubing unit 10, which is pinchable by a pinching unit 12, and a tubing holder 32 as a stationary part, which is configured to hold the compressible tubing unit 10. An additional stationary part, e.g. like the housing 28 in the first embodiment, is not necessary, however basically conceivable according to the second embodiment. Further the pinching unit 12 has a mechanical actuation unit, e.g. formed as a rotatable wheel or turning knob 158 (FIG. 19).

The medical sampling device 2 according to the second embodiment may comprise the sample container connection interface 14, the endoscope connection interface 18, the suction source connection interface 22, the mechanical actuation unit 30, and the locking key 140. When engaged and fully assembled (i.e. when the endoscope 4 is connected to the endoscope connection interface 18), the locking key 140 locks the medical sampling device 2 to the endoscope 4 in a fixed position, as described with reference to the first embodiment.

A sampling-mode and a bypass-mode can be achieved by rotating the pinching unit 12 around a rotational axis, which is essentially perpendicular to the longitudinal axis 26 and points out of the image plane in the second embodiment of the medical sampling device 2. This rotation leads to a movement of the pinching unit 12 relative to the tubing holder 32, which gradually pinches the compressible tubing unit 10 in predetermined positions. The predetermined positions are defined by the corresponding modes, namely the sampling-mode and the bypass-mode. Preferably, the rotational angle between these two modes is around 90°.

The medical sampling device 2 preferably allows just one internal degree of freedom, which is the rotation around the rotational axis.

Further, the components or parts of the medical sampling device 2 can be divided into a stationary group and a rotatable group. The stationary group comprises the tubing holder 32, the compressible tubing unit 10, the sample container attachment element 16 and the endoscope attachment element 20. The rotatable group comprises the pinching unit 12. It is added that the system borders of the internal degrees of freedom do not include external degrees of freedom, such as movements of the entire medical sampling device 2 or the system or set comprising the medical sampling device 2, the endoscope 4 and the sample container 6, e.g. during use, assembly, disassembly, etc. and movements of connection elements, which occur when the endoscope 4 is connected or disconnected, when the sample container 6 is connected or disconnected, for example.

The pinching unit 12 further comprises a narrow clamping portion 112 and a wide clamping portion 114, which are essentially arranged diametrically opposed to each other. The narrow clamping portion 112 is configured to pinch the compressible tubing unit 10 in a third position, which is located on the endoscope portion 94, shown in FIG. 9, allowing a sampling-mode. In the sampling-mode, fluid flow is just allowed from the endoscope 4 to the sample container 6 and from the sample container 6 to the suction source. The wide portion 114 is configured to pinch the compressible tubing unit 10 in a first position, which is located on the first fluid connection 80, shown in FIG. 9, and in a second position, which is located on the sample container portion 96, shown in FIG. 9. In combination, by pinching the compressible tubing part 10 in these two positions, the bypass-mode is enabled. In the bypass-mode, fluid flow is just allowed from the endoscope 4 directly to the suction source.

FIG. 19 shows an exploded perspective view of the medical sampling device 2 according to the second embodiment. The endoscope attachment element 20, the pinching unit 12, the housing 28, the suction source connection interface 22, the cover 142 and the turning knob 158 are visible.

The turning knob 158 comprises a fluted section 160 and an extrusion 162 and is adapted to be rotated in an easy way by the user. Further, the turning knob 158 is fixedly mounted on top of the pinching unit 12, so that a rotation of the turning knob 158 rotates the pinching unit 12, in order to set the sampling mode or the by-pass mode. The extrusion 162 is adapted to visually show a fluid flow direction, either to the sample container 6 or directly to the suction source 13 (see FIG. 1), according to the corresponding mode (sampling mode or by-pass mode).

In order to achieve the corresponding sampling mode or by-pass mode, the pinching unit 12 comprises a narrow clamping portion 164 and a wide clamping portion 166. In the sampling mode, just the narrow clamping portion 164 clamps/ pinches the compressible tubing unit 10 in a third pinching position 148 as described in FIG. 12 and in the by-pass mode, the wide clamping portion 166 clamps/ pinches the compressible tubing unit 10 in a first pinching position 144 and a second pinching position 146 as well as described in FIG. 12.

Further, the clamping unit 12 comprises a pin 168, which is adapted to guide the clamping unit 12 and/ or to temporarily fixate the clamping unit 12 in a rotational position, according to the desired sampling mode or by-pass mode.

The endoscope attachment element 20 is adopted to connect with its barbed end 170 to the compressible tubing unit 10 and with a side opposite to the barbed end 170 to the endoscope 4 (see FIG. 1). The endoscope 4 (see FIG. 1) is fixed/ kept in place by the locking key 140.

When assembled, the endoscope attachment element 20 sits in the aperture 172 of the housing 28 and the pinching unit 12 sits in the circular aperture 174 of the housing 28. The turning knob 158 then sits atop of the pinching unit 12.

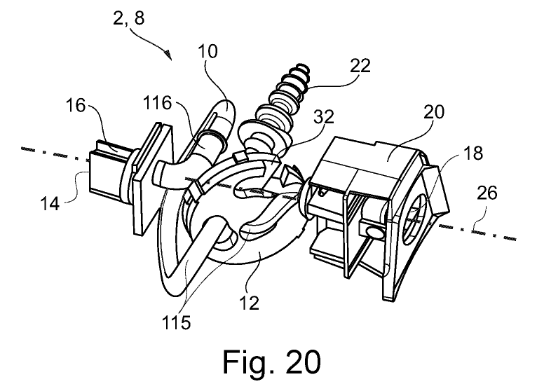

FIG. 20 shows a perspective view of the medical sampling device 2 according to a third embodiment. The functionality is similar to the first embodiment. However, a rotational axis of the pinching unit 12 is basically rotatable, advantageously allowing a broader constructional degree of freedom. The medical sampling device 2 according to the third embodiment may comprise the sample container connection interface 14, the endoscope connection interface 18, the suction source connection interface 22, the mechanical actuation unit 30, and the locking key 140. When engaged and fully assembled (i.e. when the endoscope 4 is connected to the endoscope connection interface 18), the locking key 140 locks the medical sampling device 2 to the endoscope 4 in a fixed position, as described with reference to the first embodiment.

In the third embodiment the medical sampling device 2 comprises a sample container attachment element 16 having the sample container connection interface 14, an endoscope attachment element 20 having the endoscope connection interface 18, the suction source interface 22, the longitudinal axis 26, and a pinch valve 8, wherein the pinch valve 8 comprises a compressible tubing unit 10, which is pinchable by a pinching unit 12, and a tubing holder 32, which is configured to hold the compressible tubing unit 10. A pinching plane is defined by abutting surfaces of the pinching unit 12 and the tubing holder 32. The compressible tubing unit 10 is essentially only pinched in the pinching plane. In other words, substantially no axial distance between pinching positions exists in the third embodiment.

The compressible tubing unit comprises at least three separate compressible tubes 115 and tube connectors 116, which are configured to connect the separate compressible tubes 115 to achieve functionally equal fluid connections as in the previous first and second embodiments.

A sampling-mode and a bypass-mode can be achieved by rotating the pinching unit 12 around the rotational axis. This rotation leads to a movement of the pinching unit 12 relative to the tubing holder 32, which gradually pinches the compressible tubing unit 10 in predetermined positions. The predetermined positions are defined by the corresponding modes, namely the sampling-mode and the bypass-mode. Preferably, the rotational angle between these two modes is smaller than or equal to 90°.

The medical sampling device 2 preferably allows just one internal degree of freedom, which is the rotation around the rotational axis.

Further, the components or parts of the medical sampling device 2 can be divided into a stationary group and a rotatable group. The stationary group comprises the tubing holder 32 (and potentially a housing similar to the housing 28 of the first embodiment), the compressible tubing unit 10, the sample container attachment element 16 and the endoscope attachment element 20. The rotatable group comprises the pinching unit 12. It is added that the system borders of the internal degrees of freedom do not include external degrees of freedom, such as movements of the entire medical sampling device 2 or a system or set comprising the medical sampling device 2, the endoscope 4 and the sample container 6, e.g. during use, assembly, disassembly, etc. and movements of connection elements, which occur when the endoscope 4 is connected or disconnected, when the sample container 6 is connected or disconnected, for example.

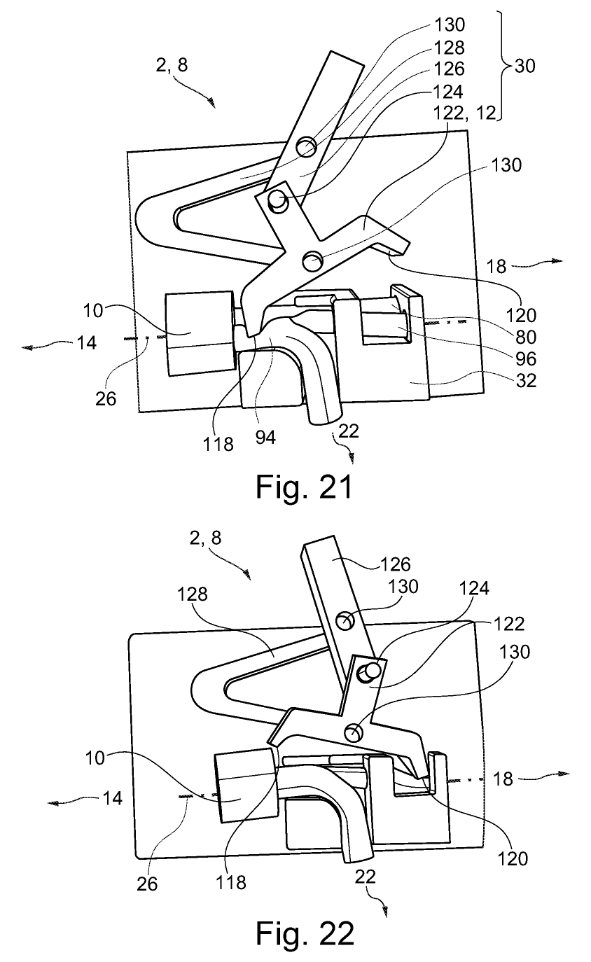

FIG. 21 shows a conceptual schematic illustration of a pinch valve prototype for a medical sampling device 2 according to a fourth embodiment in a sampling-mode. This pinch valve prototype provides a rocker switch type of operation, which may be advantageous by providing potentially user-friendly activation by shifting the actuation unit to one side or the other, which could be done with a thumb by the user with minimum effort and without changing grip on the endoscope. The pinch valve according to this embodiment may be configured to be bi-stable meaning that the pinch valve is always pinched to one side of the other, so the valve is either in bypass-mode or sampling mode.

In the fourth embodiment the medical sampling device 2 comprises a sample container attachment element 16 having a sample container connection interface 14, an endoscope attachment element 20 having an endoscope connection interface 18, a suction source interface 22, a pinch valve 8, wherein the pinch valve 8 comprises a compressible tubing unit 10, which is pinchable by a pinching unit 12, and a tubing holder 32, which is configured to hold the compressible tubing unit 10. Further, the pinching unit 12 has a mechanical actuation unit 30, which comprises a clamper 122, which acts as the pinching unit 12, an axle 124, a rocking lever 126, a spring element 128 and first and second connection interfaces 130.

The axle 124 rotatably connects the clamper 122 with the rocking lever 126, wherein the spring element is connected to the clamper 122 and the rocking lever 126 by the first and second connection interfaces 130, which preferably provide a riveted or bolted connection. The spring element 128 is configured to pull together the first and second connection interfaces 130 in a way, that a clamping force, which pinches the compressible tubing unit 10 is enough to close a corresponding fluid connection within the compressible tubing unit 10.

A sampling-mode and a bypass-mode can be achieved by rocking or shifting the pinching unit 12 around a rotational axis, which is essentially perpendicular to the longitudinal axis 26 and points out of the image plane in the fourth embodiment of the medical sampling device 2. The rotation leads to a movement of the pinching unit 12 relative to the tubing holder 32, which gradually pinches the compressible tubing unit 10 in predetermined positions. The predetermined positions are defined by the respective modes, namely the sampling-mode and the bypass-mode.

The medical sampling device 2 preferably allows just one internal degree of freedom, which is the rotation around the rotational axis.

Further, the components or parts of the medical sampling device 2 can be divided into a stationary group and a rotatable group. The stationary group comprises the tubing holder 32, the compressible tubing unit 10, the sample container attachment element 16 and the endoscope attachment element 20. The rotatable group comprises the pinching unit 12. It is added that the system borders of the internal degrees of freedom do not include external degrees of freedom, such as movements of the entire medical sampling device 2 or a system or set comprising the medical sampling device 2, the endoscope 4 and the sample container 6, e.g. during use, assembly, disassembly, etc., and movements of connection elements, which occur when the endoscope 4 is connected or disconnected, when the sample container 6 is connected or disconnected, for example.

The pinching unit 12 further comprises a narrow clamping portion 118 and a wide clamping portion 120. The narrow clamping portion 118 is configured to pinch the compressible tubing unit 10 in a third position, which is located on the endoscope portion 94, allowing a sampling-mode. In the sampling-mode, fluid flow is only allowed from the endoscope 4 to the sample container 6 and from the sample container 6 to the suction source. The wide portion 120 is configured to pinch the compressible tubing unit 10 in a first position, which is located on the first fluid connection 80, and in a second position, which is located on the sample container portion 96. In combination, by pinching the compressible tubing part 10 in these two positions, the bypass-mode is allowed. In the bypass-mode, fluid flow is only allowed from the endoscope 4 directly to the suction source.

FIG. 22 shows a perspective view of the medical sampling device 2 according to the fourth embodiment in the bypass-mode.

As can be seen, the wide portion 120 pinches the compressible tubing unit 10 in the first position, which is located on the first fluid connection 80, and in the second position, which is located on the sample container portion 96. In combination, by pinching the compressible tubing unit 10 in these two positions, the bypass-mode is allowed. In the bypass-mode, fluid flow is only allowed from the endoscope 4 directly to the suction source.

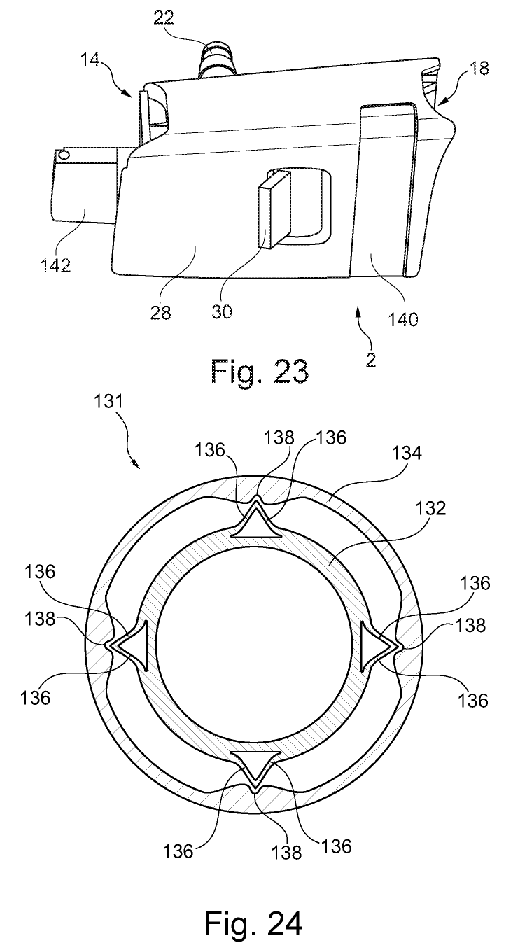

FIG. 23 shows a perspective view of the medical sampling device 2 according to the fourth embodiment. The medical sampling device 2 is in a fully assembled state and therefore compact and aesthetical appealing. In the illustrated assembled state, the housing 28, the sample container connection interface 14, the endoscope connection interface 18, the mechanical actuation unit 30, the suction source connection interface 22, the locking key 140 and the cover 142 for the sample container connection interface 14 are visible. When engaged and fully assembled (i.e. when the endoscope 4 is connected to the endoscope connection interface 18), the locking key 140 locks the endoscope 4 in the fixed position. The mechanical actuation unit 30 comprises a lever, which is adapted to be easily touched/ gripped by the user in an ergonomic way to facilitate a fast mode selection (sampling mode or by-pass mode). Further, the suction source connection interface 22 is barbed in order to securely connect to a hose. As can be seen, the cover 142 fully sheathes fluid openings of the sample container interface 14, to safeguard them from external contamination and other possible unintended influences.

FIG. 24 shows a side view of a snap-in mechanism 131. The snap-in mechanism 131 comprises a rotatable part or portion 132, a stationary part or portion 134, four spring portions 136 and four grooves 138. The grooves 138 are located within the stationary part or portion 134 and configured to accommodate the spring portions 136 in a mating-surfaces-manner. The snap-in mechanism is configured to temporarily fixate the rotatable part or portion 132 relative to the stationary part or portion 134 in such a way that an elastic deformation of the compressible tubing unit 10, when being pinched, cannot initiate a rotational movement of the rotating part by an elastic restoring force of the pinched compressible tubing unit 10. In a simple version there is only one spring portion and two grooves, one for bypass position, and one for the sample position, or one groove and two spring portions.

### IMPLEMENTATIONS

Implementations disclosed herein above and below provide systems, methods and apparatus for sampling fluids and tissue from a patient. More specifically, implementations of the present technology relate to medical sampling device, or medical sampler, interposed between a medical instrument and a sample container.

The following items are further variations and examples of the embodiments described with reference to the figures.
1. Medical sampling device (2), in particular sampler bridge, attachable to a suction connector (3) of an endoscope (4), to a sample container (6), and to a suction source (13), operable in at least a sampling-mode and a bypass-mode, the sampling-mode being a sample-collection-mode in which a specimen sample from the endoscope (4) is collected in the sample container (6), the bypass-mode being a suctioning-mode in which the specimen sample is directly guided towards the suction source, thereby bypassing the sample container (6), the medical sampling device (2) comprising a pinch valve (8) having at least a compressible tubing unit (10) and a pinching unit (12), the pinching unit (12) being configured to pinch the compressible tubing unit (10) in at least a first pinching state providing the sampling-mode and a second pinching state providing the bypass-mode.
2. The medical sampling device (2) according to item 1 further comprising a sample container connection interface (14) adapted for connection to the sample container (6), an endoscope connection interface (18) adapted for connection to the endoscope (4) and a suction source connection interface (22) adapted for connection to the suction source.
3. The medical sampling device (2) according to any one of the previous items further comprising a first axial end on which the endoscope (4) is attachable and a second axial end, which is opposite the first axial end, on which the sample container (6) is attachable, wherein a longitudinal axis (26) is formed between the first axial end and the second axial end, wherein the suction source connection interface (22) is oriented at an angle with respect to the longitudinal axis (26), preferably substantially at a right angle.
4. The medical sampling device (2) according to any one of the previous items further comprising an actuation unit configured to actuate the pinching unit (12), in particular to bring the pinching unit (12) in at least the first pinching state and the second pinching state, preferably wherein the actuation unit is a mechanical actuation unit (30), in particular is or comprises a rotatable or displaceable actuation element or portion, e.g. a lever or switch or wheel part or portion.
5. The medical sampling device (2) according to any one of the previous items, wherein the compressible tubing unit (10) is an integral part or comprises a plurality of tubes (115).
6. The medical sampling device (2) according to any one of the previous items, wherein the compressible tubing unit (10) comprises a first fluid connection (80) having a first endoscope end (82) and a first sample container end (84), and a second fluid connection (86) having a second endoscope end (88), a second sample container end (90) and a suction source end (22), wherein the pinching unit (12) is configured to pinch the compressible tubing unit (10) in the second pinching state providing the bypass-mode such that fluid flow is enabled between the second endoscope end (88) and the suction source end (22), and the pinching unit (12) is configured to pinch the compressible tubing unit (10) in the first pinching state providing the sampling-mode such that fluid flow is enabled from the first endoscope end (82) to the first sample container end (84), via the sample container (6) to the second sample container end (90) and from the second sample container end (90) to the suction source end (22).
7. The medical sampling device (2) according to any one of the previous items, wherein the pinching unit (10) is an integral part or comprises a plurality of pinching parts.
8. The medical sampling device (2) according to any one of the previous items further comprising a first axial end on which the endoscope (4) is attachable and a second axial end, which is opposite the first axial end, on which the sample container (6) is attachable, wherein a longitudinal axis (26) is formed between the first axial end and the second axial end, wherein the pinching unit (10) is a rotating part having an axis of rotation corresponding to the longitudinal axis (26).
9. The medical sampling device (2) according to any one of the items 1 to 7, further comprising a first axial end on which the endoscope (4) is attachable and a second axial end, which is opposite the first axial end, on which the sample container (6) is attachable, wherein a longitudinal axis (26) is formed between the first axial end and the second axial end, wherein the pinching unit (10) is a rotating part having an axis of rotation arranged essentially perpendicularly to the longitudinal axis (26).
10. The medical sampling device (2) according to item 9, wherein the compressible tubing unit (10) comprises a first fluid connection (80) having a first endoscope end (82) and a first sample container end (84), and a second fluid connection (86) having a second endoscope end (88), a second sample container end (90) and a suction source end (22) the pinching unit (10) formed as the rotating part comprises a first engagement end or portion (114, 120) configured and provided to pinch the first fluid connection (80) and the second fluid connection (86), and a second engagement end or portion (112, 118) configured and provided to pinch only the second fluid connection (86), wherein the first engagement end or portion (114, 120) has an engagement length greater than an engagement length of the second engagement end or portion (112, 118).
11. The medical sampling device (2) according to item 10, wherein the first engagement end or portion (114, 120) is arranged essentially diametrically opposed to the second engagement end or portion (112, 118).
12. The medical sampling device (2) according to any one of the previous items further comprising a locking or fixing mechanism, in particular a snap-in mechanism (131), configured and provided to lock or fixate the pinching unit (10) in the first pinching state and in the second pinching state.
13. The medical sampling device (2) according to any one of the previous items, wherein the compressible tubing unit (10) is made of silicone or TPU or rubber as material.
14. System comprising the medical sampling device (2) according to any one of the previous items and a sample container (6), preferably also an endoscope (4).
15. The System of item 14 comprising an endoscope (4) and a monitor (7) connectable to the endoscope.

### INTERPRETATION AND TERMINOLOGY

Any headings used herein are for organizational purposes only and shall not be used to limit the scope of the description or the claims. Furthermore, the use of certain terms is for illustration and should not be construed as limiting.

Reference to “one embodiment,” “preferred embodiment,” “an embodiment,” or “embodiments” means that a particular feature, structure, characteristic, or function described in connection with the embodiment is included in at least one embodiment and may be in more than one embodiment. Also, the appearances of the above-noted phrases in various places in the specification are not necessarily all referring to the same embodiment or embodiments.

The use of the terms “first”, “second”, “third”, “fourth”, “primary”, “secondary”, “tertiary” etc. does not imply any particular order or importance. These labels are included to identify individual elements. Furthermore, the labelling of a first element does not imply the presence of a second element and vice versa. Similarly, unless a specific order of steps or actions is required for proper operation of the method that is being described, the order and/or use of specific steps and/or actions may be modified or interchanged without departing from the scope of the claims.

Unless expressly specified otherwise, as used herein the following terms have the specified meanings:

The terms “about” or “approximately” refer to a range of measurements of a length, thickness, a quantity, time period, or other measurable values. Such range of measurements encompasses variations of +/−10 % or less, preferably +/−5 % or less, and more preferably +/−1 % or less, of and from the specified value, in so far as such variations are appropriate in order to function in the disclosed devices, systems, and techniques. The term “substantially” denotes a variability from a stated value or condition of no more than +/-10 %.

The terms “comprises”, “comprising”, “includes”, “including”, “has”, “having” or any other variation thereof, are open-ended transitional terms and should be interpreted to mean “including, but not limited to.” By contrast, “consisting” is a closed-ended term indicating that the device/method in question includes only the listed elements or steps.

The expression “consists substantially of” is a transitional phrase indicating that the device/method in question includes the listed elements or steps and potentially others that do not materially affect the basic and novel characteristics of the terms following the expression.

The use of “a” or “an” should be read to include at least one and the singular also includes the plural.

The term “or” includes an inclusive-or and a exclusive-or. For example, a condition A or B is satisfied by any one of the following: A is true (or present) and B is false (or not present), A is false (or not present) and B is true (or present), and both A and B are true (or present).

The terms/expressions “in the range” or ”between” or ”inclusive” include the endpoint values. Therefore, “in the range of A-B” includes A and B, and ”between A and B” includes A and B.

As used herein, the phrase “based on” includes both “based only on” and “based at least on.”

As used herein, the terms “couple,” “coupling,” “coupled,” and other variations of the word couple, “connect,” “connected,” and other variations of the word “connect”, and “communicatively coupled” include direct connections and indirect connections through one or more intermediary parts or devices. Communicatively coupled includes wireless connections. “Coupled” is not limited to a connection of two (a couple) of items. In the context of passages and channels, the term “connected” denotes that a fluid can pass from one to the other and may be substituted by the expression “fluidly connected” or “fluidly coupled” to indicate that fluidly connected components allow fluid to pass between the components without requiring that the components physically touch. A passage or channel may abut another passage or channel, in which case the connection is direct, or may be separated from another passage or channel by, for example, a fluid seal.

As used herein, the term “determining” encompasses a wide variety of actions and, therefore, “determining” can include calculating, computing, processing, deriving, investigating, looking up (e.g., looking up in a table, a database or another data structure), ascertaining and the like.

The term “distal” is defined to be in the direction toward the patient, and the term “proximal” is defined to be in the direction away from the patient. For example, the handle is at the proximal end of an endoscope and the bending section is located distally from the handle.

The term “integral” denotes a structural relationship in which components cooperate as a whole through permanent formation or permanent joining, regardless of whether the components originate from a single manufacturing operation or from multiple manufacturing operations followed by permanent union. Thus, integral excludes components merely juxtaposed, detachable, friction fit, removable, entirely separable, held together during use, or in mere spatial proximity. The term “integral formed” refers to a single-piece structure in which the components are formed together as a single piece rather than joined from separately manufactured components. Thus, integrally formed structures include structures formed or molded, and may include structures made by 3D printing and EDM machining. An integrally formed structure may be formed in a single molding/extrusion/casting operation. In contrast to integrally formed embodiments, integrally joined embodiments may be permanently assembled from multiple discrete components and joined by welding, ultrasonic welding, brazing, bonding, and similar techniques.

The term “videoscope” includes medical devices insertable into a patient and including an image sensor to capture distal or lateral views from the distal end of the videoscope. Videoscopes include endoscopes, laryngoscopes, and endotracheal tubes.

The term “endoscope” is a general term that includes procedure-specific devices including arthroscopes, bronchoscopes, cardioscopes, colonoscopes, cystoscopes, duodenoscopes, gastroscopes, hysteroscopes, laparoscopes, nasopharyngoscopes, sinuscopes, uroscopes, and urethroscopes.

The term “plurality” denotes two or more. For example, a plurality of components indicates two or more components.

The term “positioning interface,” or “interface,” refers to a component that functions to control the position of the insertion cord. The handle is an example of a positioning interface and, unless stated otherwise, the terms are used interchangeably. Alternatively, a different positioning interface can be provided that is connected to the insertion cord and is detachably connected to a robotic arm. In a further alternative, the positioning interface is detachably connected to the insertion cord and is therefore reusable. In this example, the positioning interface may be affixed to a robotic arm. The insertion cord thus extends from the robotic arm, and the endoscope is detachable from the robotic arm. The robotic arm responds to signals, including voice commands from an operator, to rotate, translate, and otherwise position the proximal end of the insertion cord, as an operator would do manually. The positioning interface can include control actuators, including manual control actuators. Alternatively, or additionally, control actuators can be provided in or on the robotic arm or by the robotic system including the robotic arm, thereby potentially reducing the cost of the endoscope. Example control actuators include single axis actuators, including linear motion actuators. A linear motion actuator may comprise a threaded rod coupled to a threaded nut portion, in which a motor rotates the rod to translate the nut portion. The control actuators may comprise steering, vacuum/suction, recording, and other controls, e.g. knobs, levers, buttons, and the like, used to steer the tip part of the insertion cord, to engage vacuum, to capture images or videos, etc.

### LIST OF REFERENCE NUMBERS

2 medical sampling device

3 suction connector

4 endoscope

5 working channel

6 sample container

7 suction button

8 pinch valve

9 monitor

10 compressible tubing unit

11 cable

12 pinching unit

13 suction source

14 sample container connection interface

15 insertion cord

16 sample container attachment element

17 hose

18 endoscope connection interface

20 endoscope attachment element

22 suction source connection interface/ suction source end

26 longitudinal axis

28 housing

30 mechanical actuation unit

32 tubing holder

34 first tubing connection

36 second tubing connection

38 tubing connection

40 housing endoscope opening

42 housing suction source opening

44 first sample container opening

46 second sample container opening

48 curved portion

50 straight portion

52 actuation unit opening

54 tubing holder recess

56 tubing holder protrusion

58 first tubing holder endoscope opening

60 second tubing holder endoscope opening

62 suction source opening

64 second tubing holder sample container opening

66 clamping unit recess

68 first clamping portion (first position)

70 second clamping portion (second position)

72 curved portion

74 straight portion

76 clamping claw

78 combined first and second position portion

80 first fluid connection

82 first endoscope end

84 first sample container end

86 second fluid connection

88 second endoscope end

90 second sample container end

92 suction source end

94 endoscope portion

96 sample container portion

98 suction source portion

99 common point

100 passage (clamped)

102 passage (open)

104 passage (open)

106 passage (clamped)

108 passage (clamped)

110 passage (open)

111 arrow

112 narrow clamping portion/ second engagement end/ portion

114 wide clamping portion/ first engagement end/ portion

115 compressible tubes, plurality of tubes

116 tube connectors

118 narrow clamping portion/ second engagement end/ portion

120 wide clamping portion/ first engagement end/ portion

122 clamper

124 axle (with through hole)

126 rocking lever

128 spring element

130 first and second through holes

131 snap-in mechanism

132 rotating part/ portion

134 stationary part/ portion

136 spring portion

138 groove

140 locking key

142 cover

144 first pinching position

146 second pinching position

148 third pinching position

150 fluid way

152 fluid way

154 fluid way

156 fluid way

158 turning knob

160 fluted section

162 extrusion

164 narrow clamping portion

166 wide clamping portion

168 pin

170 barbed end

172 aperture

174 circular aperture

1. A medical sampler attachable to a suction connector of an endoscope, to a sample container, and to a suction source, the medical sampler comprising:
a pinch valve having an endoscope end and a sample container end, the pinch valve comprising:
a first conduit selectively compressible between a closed configuration and an open configuration, the closed configuration occluding fluid flow and the open configuration permitting fluid flow;
a suction opening;
a second conduit extending from the sample container end to the suction opening, the second conduit being selectively compressible between a closed configuration and an open configuration, the closed configuration occluding fluid flow and the open configuration permitting fluid flow;
a third conduit extending from the endoscope end to the suction opening, the third conduit being selectively compressible between a closed configuration and an open configuration, the closed configuration occluding fluid flow and the open configuration permitting fluid flow; and
a pinching unit configured place the pinch valve in a sampling mode or a bypass mode,
wherein in the sampling-mode of the pinch valve the first conduit and the second conduit are, concurrently, in the open configuration and the third conduit is in the closed configuration, whereby suction applied at the suction opening creates suction through the second conduit and the first conduit but not the third conduit, and
wherein in the bypass-mode of the pinch valve the first conduit and the second conduit are, concurrently, in the closed configuration and the third conduit is in the open configuration, whereby the suction applied at the suction opening creates suction through the third conduit but not through the second conduit and not through the first conduit.

2. The medical sampler of claim 1, wherein the first conduit, the second conduit, and the third conduit are integrally formed.

3. The medical sampler of claim 2, further comprising a tube extending from the endoscope end to the sample container end, the tube including the second conduit and the third conduit, the tube further comprising the suction opening in a wall of the tube, the suction opening being located between the second conduit and the third conduit.

4. The medical sampler of claim 1, wherein the pinching unit comprises:
a housing extending from the endoscope end to the sample container end and comprising a longitudinal axis extending from the endoscope end to the sample container end; and
a drum rotatable in the housing to switch the pinch valve between the bypass-mode and the sampling-mode.

5. The medical sampler of claim 4, wherein the drum comprises a rotation axis that is substantially parallel to the longitudinal axis.

6. The medical sampler of claim 5, wherein the drum comprises a first conduit opening and a second conduit opening, the first conduit traversing the first conduit opening and the second conduit traversing the second conduit opening, wherein the first conduit opening and the second conduit opening each comprises a perimeter comprising:
(i) a first end portion defined by a semicircular arc;
(ii) a slot portion extending from the semicircular arc and defined by a first boundary segment and a second boundary segment, the first boundary segment having a constant curvature having a first radius, and the second boundary segment having a constant curvature having a second radius smaller than the first radius; and
(iii) a terminal portion comprising a straight portion and a transition portion connecting the straight portion with the second boundary segment, the transition portion transitioning the second boundary segment, in a continuous manner, from the second radius to a smaller radius culminating in a pointed or tapered end.

7. The medical sampler of claim 5, further comprising a tube extending from the endoscope end to the sample container end, the tube including the second conduit, the third conduit and the suction opening in a wall of the tube, wherein the first conduit and the tube are integrally formed.

8. The medical sampler of claim 4, wherein the drum comprises a rotation axis that is substantially perpendicular to the longitudinal axis.

9. The medical sampler of claim 1, further comprising a suction tube connected to the suction opening and extending at an angle of between 45 and 135 degrees with respect to the longitudinal axis.

10. The medical sampler of claim 1, wherein the first conduit, the second conduit, and the third conduit are comprised of a thermoplastic elastomer configured to enable each of the first conduit, the second conduit, and the third conduit to undergo elastic deformation in response to an applied force and to substantially return to its original shape upon removal of the applied force, and wherein the first conduit, the second conduit, and the third conduit each comprises a wall of a thickness sufficient to prevent collapse of the first conduit, the second conduit, and the third conduit, respectively, responsive to the suction applied at the suction opening.

11. The medical sampler of claim 1, wherein the pinching unit comprises:
a housing extending from the endoscope end to the sample container end, the housing comprising a first compression portion, a second compression portion, and a third compression portion; and
a drum rotatable in the housing and comprising a first compression portion, a second compression portion, and a third compression portion,
wherein in the bypass-mode of the pinch valve the first compression portion of the housing and the first compression portion of the drum compress the first conduit in a radial direction to occlude fluid flow through the first conduit, and the second compression portion of the housing and the second compression portion of the drum compress the second conduit in a radial direction to occlude fluid flow through the second conduit, and
wherein in the sampling-mode of the pinch valve the third compression portion of the housing and the third compression portion of the drum compress the third conduit in a radial direction to occlude fluid flow through the third conduit.

12. The medical sampler of claim 11, further comprising a drum actuator configured to rotate the drum and thus switch the pinch valve between the bypass-mode and the sampling-mode.

13. The medical sampler of claim 12, wherein the housing comprises a sample container connection interface configured to physically and directly connect the sample container, an endoscope connection interface configured to physically and directly connect the housing to the endoscope and a suction source connection interface configured to fluidly connect with the suction source.

14. The medical sampler of claim 1, further comprising a snap-in mechanism configured and provided to lock or fixate the pinch valve in the bypass-mode or in the sampling mode.

15. A system comprising:
the medical sampler of claim 1;
the sample container; and
the endoscope.

16. The System of claim 15, further comprising a monitor connectable to the endoscope.
